(19) United States
(12) Patent Application Publication
(10) Pub. No.: US 2023/0113378 A1
(43) Pub. Date: Apr. 13, 2023

(54) Methods Of Treating Multiple Sclerosis

(71) Applicant: Actelion Pharmaceuticals Ltd, Allschwil (CH)

(72) Inventors: Krista Spiller, Philadelphia, PA (US); Anindya Bhattacharya, San Diego, CA (US); Tatiana Sidorenko, Allschwil (CH); Janice Wong, Winchester, MA (US)

(21) Appl. No.: 17/962,641

(22) Filed: Oct. 10, 2022

Related U.S. Application Data
(60) Provisional application No. 63/254,428, filed on Oct. 11, 2021.
(60) Provisional application No. 63/336,166, filed on Apr. 28, 2022.
(60) Provisional application No. 63/342,803, filed on May 17, 2022.

(51) Int. Cl. A61K 39/215; A61K 31/33; A61K 39/145; A61K 31/426; A61P 31/14; A61P 37/02

(52) U.S. Cl. CPC A61K 39/215; A61K 31/33; A61K 39/145; A61K 31/426; A61P 31/14; A61P 37/02; A61K 2039/5252

(57) ABSTRACT

The disclosure relates to methods of treating multiple sclerosis and maintaining or maximizing vaccine effectiveness. In certain aspects, the methods comprise administrating ponesimod, administering a vaccine, and interrupting the administration of the ponesimod.

### CROSS-REFERENCE TO RELATED APPLICATIONS

This application claims the benefit of U.S. Provisional Patent Application No. 63/342,803, filed May 17, 2022, U.S. Provisional Patent Application No. 63/336,166, filed Apr. 28, 2022, and U.S. Provisional Patent Application No. 63/254,428, filed Oct. 11, 2021, the entireties of which are incorporated herein by reference.

### TECHNICAL FIELD

The present disclosure relates to methods of treating multiple sclerosis, including methods for maintaining or maximizing vaccine effectiveness in patients being administered ponesimod.

### BACKGROUND

Multiple sclerosis (MS) is a chronic autoimmune inflammatory disease of the central nervous system affecting over 2.5 million people worldwide. The disease is characterized by demyelination and axonal loss leading to neurological impairment and severe disability. The two main subtypes of MS at disease onset are relapsing forms of MS (RMS) which represent 85% of MS patients and include relapsing-remitting disease (RRMS), clinically isolated syndrome, and active secondary progressive disease; secondary progressive MS is a phenotype that in many cases follows RMS; and primary progressive MS (PPMS) which affects only 15% of MS patients at disease onset. Ponesimod is a newly approved treatment for Relapsing Multiple Sclerosis in adults. It is a selective sphingosine 1-phosphate receptor 1 (SIP1) modulator that blocks the capacity of lymphocytes to egress from lymph nodes. Clinical data suggest that SIP receptor modulators may impact the humoral immune response to non-live vaccines, including those for COVID-19 and influenza.

For instance, two studies (Achiron A, Mandel M et al. Humoral immune response to COVID-19 mRNA vaccine in patients with multiple sclerosis treated with high-efficacy disease-modifying therapies. Ther Adv Neurol Disord 2021, Vol. 14: 1-8; Achiron A, Dolev M et al. COVID-19 vaccination in patients with multiple sclerosis: What we have learnt by February 2021. Multiple Sclerosis Journal 1-7. Mult Scler. 2021 Apr. 15) exploring the safety of the mRNA-based BNT162b2 (BioNTech and Pfizer) COVID-19 vaccine in MS patients (N=555) and its efficacy in MS patients (n=125) treated with high-efficacy disease-modifying therapies (DMT) such as cladribine, fingolimod and ocrelizumab have recently been published. The data is shown in FIG. 1. While in the safety cohort of MS patients, vaccination appeared to be safe and well tolerated, in patients receiving continuous fingolimod treatment, the vaccine failed to induce a humoral immune response in 25 of 26 patients as assessed by SARS-CoV-2 IgG antibodies in blood samples collected 1 month after the 2^{nd} dose of the vaccine.

These data do not clarify if the fingolimod-treated patients who received COVID-19 vaccine are still at risk of developing COVID-19 disease and the vaccine-specific memory T- or B-cell responses were not assessed. This is of importance especially in MS patients who did not develop humoral immunity, as the absence of specific S1 spike antibodies does not necessarily mean absence of a protective adaptive immune response against SARS-CoV-2.

Further data from another study indicates that the cellular immune response plays a relevant role in the immune response against SARS-CoV-2 (Le Bert N, Clapham H E et al, Highly functional virus-specific cellular immune response in asymptomatic SARS-CoV-2 infection. J Exp Med (2021) 218 (5): e20202617). These observations are consistent with the reduced vaccination response that was observed previously with fingolimod and siponimod to the influenza vaccine. In the context of the COVID-19 pandemic and vaccination, an open question is whether patients taking ponesimod can respond to a novel vaccine.

### SUMMARY

In some aspects, the present disclosure is directed to methods of treating multiple sclerosis and maintaining or maximizing vaccine effectiveness, comprising administrating ponesimod to the patient in need thereof and administering a vaccine to the patient.

In some aspects, the present disclosure is directed to methods of treating multiple sclerosis and maintaining vaccine effectiveness, comprising administrating ponesimod to a patient in need thereof, and administering a vaccine to the patient without discontinuing or interrupting the administration of the ponesimod.

In some aspects, the present disclosure is directed to methods of treating multiple sclerosis and maximizing vaccine effectiveness, comprising administrating ponesimod to a patient in need thereof, administering a vaccine to the patient, and discontinuing or interrupting the administration of the ponesimod for a period of time. In particular embodiments, the ponesimod administration is interrupted prior to vaccination and resumed after a certain period of time following the vaccine administration.

In other aspects, the present disclosure is directed to ponesimod for use in a method of treating multiple sclerosis and maintaining or maximizing vaccine effectiveness, comprising administrating ponesimod to a patient in need thereof and administering a vaccine to the patient.

In other aspects, the present disclosure is directed to ponesimod for use in a method of treating multiple sclerosis and maintaining vaccine effectiveness, comprising administrating ponesimod to a patient in need thereof, and administering a vaccine to the patient without discontinuing or interrupting the administration of the ponesimod.

In other aspects, the present disclosure is directed to ponesimod for use in a method of treating multiple sclerosis and maximizing vaccine effectiveness, comprising administrating ponesimod to a patient in need thereof, administering a vaccine to the patient, and discontinuing or interrupting the administration of the ponesimod for a period of time. In particular embodiments, the ponesimod administration is interrupted prior to vaccination and resumed after a certain period of time following the vaccine administration.

In other aspects, the present disclosure is directed to the use of ponesimod in the manufacture of a medicament for treating multiple sclerosis and maintaining or maximizing vaccine effectiveness, wherein said medicament is adapted to be administered using a regimen comprising administrating ponesimod to a patient in need thereof and administering a vaccine to the patient.

In other aspects, the present disclosure is directed to the use of ponesimod in the manufacture of a medicament for treating multiple sclerosis and maintaining vaccine effectiveness, wherein said medicament is adapted to be administered using a regimen comprising administrating ponesimod to a patient in need thereof, and administering a vaccine to the patient without discontinuing or interrupting the administration of the ponesimod.

In other aspects, the present disclosure is directed to the use of ponesimod in the manufacture of a medicament for treating multiple sclerosis and maximizing vaccine effectiveness, wherein said medicament is adapted to be administered using a regimen comprising administrating ponesimod to a patient in need thereof, administering a vaccine to the patient, and discontinuing or interrupting the administration of the ponesimod for a period of time. In particular embodiments, the ponesimod administration is interrupted prior to vaccination and resumed after a certain period of time following the vaccine administration.

In some aspects, the vaccine is a COVID-19 vaccine. In some aspects, the vaccine is a flu vaccine. In some aspects, the vaccine is a non-live COVID-19 vaccine. In some aspects, the vaccine is a non-live flu vaccine.

The disclosure is also directed to a pharmaceutical product comprising ponesimod, wherein the pharmaceutical product is packaged and the package includes instructions for treating multiple sclerosis and maintaining or maximizing vaccine effectiveness.

### BRIEF DESCRIPTION OF THE DRAWINGS

FIG. 1 depicts a distribution chart of the effects of COVID-19 mRNA vaccines on humoral response in MS patients receiving certain disease-modifying therapies (DMT).

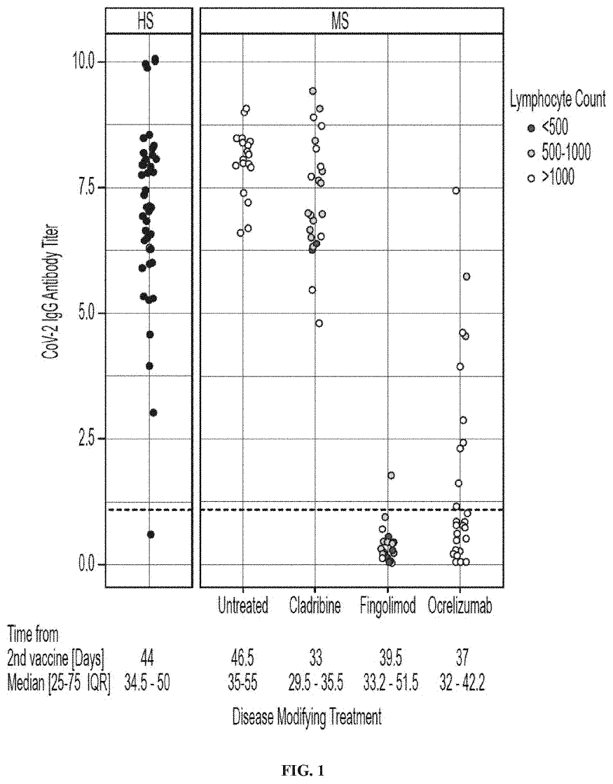

FIG. 2 depicts a distribution chart of the effects of COVID-19 mRNA vaccines on humoral response in MS patients receiving ponesimod.

FIG. 3 depicts a table breaking down the groups of female BALB/c mice that were maintained on different oral ponesimod regimens in connection with Example 3.

FIG. 4 depicts a bar graph for Groups 2 and 3 of a pharmacokinetic analysis regarding drug levels by Day 5 in connection with Example 3.

FIG. 5 depicts graphs of % inhibition vs. dilution at 4-week (left graph) and 8-week (right graph) post-immunization in connection with Example 3.

FIG. 6 depicts a graph of SARS-CoV-2 N binding antibody concentration (ELISA Units (EU)/mL): boxplot of the actual values pre- and post-vaccination by vaccine type for 1^{st} vaccine regimen.

FIG. 7 depicts a graph of SARS-CoV-2 S binding antibody concentration (ELISA Units (EU)/mL): boxplot of the actual values pre- and post-vaccination by vaccine type for 1^{st} vaccine regimen.

### DETAILED DESCRIPTION OF ILLUSTRATIVE EMBODIMENTS

In the present disclosure the singular forms “a”, “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” or “substantially” it will be understood that the particular value forms another embodiment. In general, use of the term “about” or “substantially” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about” or “substantially”. In other cases, the gradations used in a series of values may be used to determine the intended range available to the term “about” or “substantially” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the disclosure which are, for clarity, described herein in the context of separate embodiments, may also be provided in combination in a single embodiment. That is, unless obviously incompatible or excluded, each individual embodiment is deemed to be combinable with any other embodiments and such a combination is considered to be another embodiment. Conversely, various features of the disclosure that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation. Finally, while an embodiment may be described as part of a series of steps or part of a more general structure, each said step may also be considered an independent embodiment in itself.

It should be understood that references herein to methods of treatment (e.g. methods for treating multiple sclerosis and maximizing or maintaining vaccine effectiveness in a patient in need thereof) using ponesimod should also be interpreted as references to:
ponesimod or formulations thereof for use in methods of treatment (e.g., methods for treating multiple sclerosis and maximizing or maintaining vaccine effectiveness in a patient in need thereof); and/or
the use of ponesimod or formulations thereof in the manufacture of a medicament for treating multiple sclerosis and maximizing or maintaining vaccine effectiveness in a patient in need thereof.

Vaccines are an important class of medications that are frequently used for treating or preventing various diseases, such as infectious diseases. Patients having MS can receive one or more vaccines prior to, during, or after treatment with ponesimod.

In certain embodiments, methods are provided to treat MS and maintain or maximize vaccine effectiveness. In certain embodiments, methods are provided to treat MS and maintain or maximize vaccine effectiveness, comprising administrating ponesimod to the patient in need thereof and administering a vaccine to the patient.

In certain embodiments, methods are provided to treat MS and maintain vaccine effectiveness, comprising administrating ponesimod to the patient in need thereof, and administering a vaccine to the patient without discontinuing or interrupting the administration of the ponesimod. In this context, maintaining vaccine effectiveness refers to eliciting an immune response (e.g., humoral and/or cellular), even if the immune response may not be the same as the response in the absence of treatment with ponesimod.

In certain embodiments, methods are provided to treat MS and maximize vaccine effectiveness, comprising administrating ponesimod to the patient in need thereof, administering a vaccine to the patient, and discontinuing or interrupting the administration of the ponesimod for a period of time. In particular embodiments, the ponesimod administration is interrupted prior to vaccination and resumed after a certain period of time following the vaccine administration. In this context, the interruption of the scheduled/prescribed ponesimod administration would maximize vaccine effectiveness by resulting in a response that is greater than the response in the absence of such interruption. As described herein, the interruption of the scheduled/prescribed ponesimod administration can be implemented before and/or after vaccine administration. For example, the interruption can be implemented one week before a scheduled vaccine administration and continue three weeks after such scheduled administration. Following the interruption period, the previously scheduled/prescribed dosing regimen for ponesimod can be resumed or a re-initiation dosing regimen can be implemented.

The immune response can be assessed by a physician or other medical professional/personnel with the use of tests/assays directed to measuring an immune response (e.g., humoral or cellular). In certain embodiments, an immune response involves a positive or increased antibody concentration or an antibody response with low inhibition titers. As a non-limiting example, and as reflected in the examples, a response can correspond to seroconversion in case of negative pre-vaccination antibody testing, or an antibody concentration increase, such as, for example, a 4-fold antibody concentration increase, in case of positive pre-vaccination antibody result.

In certain embodiments, an immune response varies by lymphocyte count. For example, in certain embodiments, a patient may have a pre-vaccination lymphocyte count of <500 cells/mm³; or a pre-vaccination lymphocyte count 500 cells/mm³. In particular embodiments, a patient having a pre-vaccination lymphocyte count 500 cells/mm³ has a higher likelihood of having a more pronounced immune response than a patient having a pre-vaccination lymphocyte count <500 cells/mm³. In certain embodiments, the methods disclosed herein of maintaining or maximizing vaccine effectiveness are directed to patients having a pre-vaccination lymphocyte count 500 cells/mm³. In other embodiments, a ponesimod-treated patient has a pre-vaccination lymphocyte count 500 cells/mm³ and/or is administered an mRNA vaccine. In other embodiments, a ponesimod-treated patient has a pre-vaccination lymphocyte count 500 cells/mm³ and/or is administered a viral vector vaccine. In other embodiments, a ponesimod-treated patient has a pre-vaccination lymphocyte count 500 cells/mm³ and/or is administered an inactivated vaccine. In other embodiments, a ponesimod-treated patient has a pre-vaccination lymphocyte count 500 cells/mm³ and/or is administered a protein subunit vaccine. In other embodiments, a ponesimod-treated patient has a pre-vaccination lymphocyte count 500 cells/mm³ and/or is administered a vaccine different than an mRNA vaccine, for example, a viral vector vaccine, an inactivated vaccine, or protein subunit vaccine, as a first vaccination dose, and an mRNA vaccine as a second vaccination dose. In certain embodiments, the methods disclosed herein of maintaining or maximizing vaccine effectiveness are directed to patients having a pre-vaccination lymphocyte count <500 cells/mm³. In certain embodiments, a ponesimod-treated patient has a pre-vaccination lymphocyte count <500 cells/mm³ and/or is administered an mRNA vaccine. In other embodiments, a ponesimod-treated patient has a pre-vaccination lymphocyte count <500 cells/mm³ and/or is administered a viral vector vaccine. In other embodiments, a ponesimod-treated patient has a pre-vaccination lymphocyte count <500 cells/mm³ and/or is administered an inactivated vaccine. In other embodiments, a ponesimod-treated patient has a pre-vaccination lymphocyte count <500 cells/mm³ and/or is administered a protein subunit vaccine. In other embodiments, a ponesimod-treated patient has a pre-vaccination lymphocyte count <500 cells/mm³ and/or is administered vaccine different than an mRNA vaccine, for example, a viral vector vaccine, an inactivated vaccine, or protein subunit vaccine, as a first vaccination dose, and an mRNA vaccine as a second vaccination dose. Accordingly, the methods disclosed herein of maintaining or maximizing vaccine effectiveness, comprise, for example, identifying the vaccine type and/or pre-vaccination lymphocyte count of the patient.

In certain embodiments, the vaccine is a COVID-19 vaccine. In certain embodiments, the vaccine is a flu vaccine. In certain embodiments, the vaccine is a non-live COVID-19 vaccine. In certain embodiments, the vaccine is a non-live flu vaccine. In certain embodiments, the vaccine is a live COVID-19 vaccine. In certain embodiments, the vaccine is a live flu vaccine.

In certain embodiments, the MS treatments described herein have no effect on vaccine efficacy. In other embodiments, the MS treatments may result in a decrease to vaccine effectiveness compared to effectiveness in the absence of treatment with ponesimod, but still elicits an immune response (e.g., humoral and/or cellular).

In some embodiments, the patient to receive the vaccine is treatment naïve with respect to ponesimod, such that the ponesimod is first administered after administration of the vaccine. In this situation, the patient may wait to start the initial treatment regimen with ponesimod until a period of time elapses to allow for an antibody response to the vaccine, e.g., a positive or increased antibody concentration or an antibody response with low inhibition titers.

In some embodiments, the patient to receive the vaccine is currently being treated with ponesimod, such that the ponesimod is administered prior to administration of the vaccine or concurrently with the vaccine.

In some embodiments, the methods comprise administering ponesimod to a patient in need thereof, administering a vaccine to the patient, and interrupting the administration of the ponesimod for a period of time. In particular embodiments, the ponesimod administration is interrupted prior to vaccination and resumed after a certain period of time following the vaccine administration.

In some embodiments, the methods comprise interrupting the ponesimod administration prior to the vaccine administration. In some embodiments, the ponesimod administration is interrupted for a period of time to allow for an antibody response e.g., a positive or increased antibody concentration or an antibody response with low inhibition titers. In particular embodiments, the ponesimod administration is interrupted prior to vaccination and resumed after a certain period of time following the vaccine administration. The amount of time for the interruption of the ponesimod administration prior to and/or after the vaccine administration may depend on type of vaccine being given and/or the expected time for a maximum immune response.

In some embodiments, the methods comprise interrupting the ponesimod administration for about two weeks. For example, the methods comprise interrupting the ponesimod administration for about two weeks starting from about one week prior to the administration of the vaccine (and continuing for one week after the vaccine administration for a total of two weeks).

In some embodiments, the methods comprise interrupting the ponesimod administration for about three weeks. For example, the methods comprise interrupting the ponesimod administration for about three weeks starting from about one week prior to the administration of the vaccine (and continuing for two weeks after the vaccine administration for a total of three weeks). For example, the methods comprise interrupting the ponesimod administration for about three weeks starting from about two weeks prior to the administration of the vaccine (and continuing for one week after the vaccine administration for a total of three weeks).

In some embodiments, the methods comprise interrupting the ponesimod administration for about four weeks. For example, the methods comprise interrupting the ponesimod administration for about four weeks starting from about one week prior to the administration of the vaccine (and continuing for three weeks after the vaccine administration for a total of four weeks). For example, the methods comprise interrupting the ponesimod administration for about four weeks starting from about two weeks prior to the administration of the vaccine (and continuing for two weeks after the vaccine administration for a total of four weeks).

In some embodiments, the methods comprise interrupting the ponesimod administration for about five weeks. For example, the methods comprise interrupting the ponesimod administration for about five weeks starting from about one week prior to the administration of the vaccine (and continuing for four weeks after the vaccine administration for a total of five weeks). For example, the methods comprise interrupting the ponesimod administration for about five weeks starting from about two weeks prior to the administration of the vaccine (and continuing for three weeks after the vaccine administration for a total of five weeks).

In some embodiments, the methods comprise interrupting the ponesimod administration for about six weeks. For example, the methods comprise interrupting the ponesimod administration for about six weeks starting from about one week prior to the administration of the vaccine (and continuing for five weeks after the vaccine administration for a total of six weeks). For example, the methods comprise interrupting the ponesimod administration for about six weeks starting from about two weeks prior to the administration of the vaccine (and continuing for four weeks after the vaccine administration for a total of six weeks).

In some embodiments, the methods comprise interrupting the ponesimod administration for about seven weeks. For example, the methods comprise interrupting the ponesimod administration for about seven weeks starting from about one week prior to the administration of the vaccine (and continuing for six weeks after the vaccine administration for a total of seven weeks). For example, the methods comprise interrupting the ponesimod administration for about seven weeks starting from about two weeks prior to the administration of the vaccine (and continuing for five weeks after the vaccine administration for a total of seven weeks).

In some embodiments, the methods comprise interrupting the ponesimod administration for about eight weeks. For example, the methods comprise interrupting the ponesimod administration for about eight weeks starting from about one week prior to the administration of the vaccine (and continuing for seven weeks after the vaccine administration for a total of eight weeks). For example, the methods comprise interrupting the ponesimod administration for about eight weeks starting from about two weeks prior to the administration of the vaccine (and continuing for six weeks after the vaccine administration for a total of eight weeks).

In some embodiments, the methods comprise interrupting the ponesimod administration for about nine weeks. For example, the methods comprise interrupting the ponesimod administration for about nine weeks starting from about one week prior to the administration of the vaccine (and continuing for eight weeks after the vaccine administration for a total of nine weeks). For example, the methods comprise interrupting the ponesimod administration for about nine weeks starting from about two weeks prior to the administration of the vaccine (and continuing for seven weeks after the vaccine administration for a total of nine weeks).

In some embodiments, the methods comprise interrupting the ponesimod administration for about ten weeks. For example, the methods comprise interrupting the ponesimod administration for about ten weeks starting from about one week prior to the administration of the vaccine (and continuing for nine weeks after the vaccine administration for a total of ten weeks). For example, the methods comprise interrupting the ponesimod administration for about ten weeks starting from about two weeks prior to the administration of the vaccine (and continuing for eight weeks after the vaccine administration for a total of ten weeks).

In some embodiments, the methods comprise interrupting the ponesimod administration for about eleven weeks. For example, the methods comprise interrupting the ponesimod administration for about eleven weeks starting from about one week prior to the administration of the vaccine (and continuing for ten weeks after the vaccine administration for a total of eleven weeks). For example, the methods comprise interrupting the ponesimod administration for about eleven weeks starting from about two weeks prior to the administration of the vaccine (and continuing for nine weeks after the vaccine administration for a total of eleven weeks).

In some embodiments, the methods comprise interrupting the ponesimod administration for about twelve weeks. For example, the methods comprise interrupting the ponesimod administration for about twelve weeks starting from about one week prior to the administration of the vaccine (and continuing for eleven weeks after the vaccine administration for a total of twelve weeks). For example, the methods comprise interrupting the ponesimod administration for about twelve weeks starting from about two weeks prior to the administration of the vaccine (and continuing for ten weeks after the vaccine administration for a total of twelve weeks).

In preferred embodiments, the methods comprise interrupting the ponesimod administration for about five weeks. For example, preferred methods comprise interrupting the ponesimod administration for about five weeks starting from about one week prior to the administration of the vaccine (and continuing for four weeks after the vaccine administration for a total of five weeks). In preferred embodiments, the methods comprise interrupting the ponesimod administration for about four weeks. For example, preferred methods comprise interrupting the ponesimod administration for about four weeks starting from about one week prior to the administration of the vaccine (and continuing for three weeks after the vaccine administration for a total of four weeks). In preferred embodiments, the methods comprise interrupting the ponesimod administration for about three weeks starting from about one week prior to the administration of the vaccine (and continuing for two weeks after the vaccine administration for a total of three weeks).

In some embodiments, the period of time of interruption of ponesimod can be, for example, at least one, two, three, four, five, six, seven, eight, nine, ten, eleven, twelve, thirteen or fourteen days prior to the administration of the vaccine. In some embodiments, the period of time of interruption of ponesimod can be, for example, at least two, three, four, five, six, seven, eight, nine, ten, eleven or twelve weeks starting from about one or two weeks prior to the administration of the vaccine.

In some embodiments, at the end of the interrupted time period, the ponesimod treatment is resumed. For example, in some embodiments, the ponesimod treatment is resumed one, two, three, four, five, six, seven, eight, nine, ten, eleven, or twelve weeks after the vaccine administration.

In some embodiments, the scheduled/prescribed daily dose of ponesimod comprises about 15 to about 25 mg of ponesimod, and such a dose would be administered immediately following the interrupted period. For example, in certain embodiments, the daily dose of resumed ponesimod treatment comprises about 15 mg, about 16 mg, about 17 mg, about 18 mg, about 19 mg, about 20 mg, about 21 mg, about 22 mg, about 23 mg, about 24 mg, or about 25 mg of ponesimod. In certain embodiments, the daily dose of resumed ponesimod treatment comprises about 20 mg of ponesimod.

In some embodiments, about 20 mg of ponesimod is administered orally once daily for resuming the ponesimod treatment after vaccine administration. In other embodiments, the resumed ponesimod treatment after vaccine administration comprises an up-titration, followed by a daily maintenance dose of ponesimod. In other embodiments, the resumed ponesimod treatment after vaccine administration comprises an up-titration regimen as described herein, followed by a daily maintenance dose of ponesimod.

In some embodiments, the disclosure is directed to a pharmaceutical product comprising ponesimod, wherein the pharmaceutical product is packaged, and the package includes instructions for treating MS and maintaining or maximizing vaccine effectiveness as described herein.

### Flu Vaccine

In some embodiments, the patient to receive a flu vaccine is currently being treated with ponesimod, such that the ponesimod is administered prior to administration of the flu vaccine or concurrently with the flu vaccine.

In some embodiments, the methods comprise administering ponesimod to a patient in need thereof and administering a flu vaccine to the patient without interrupting the administration of the ponesimod for a period of time. In certain of these embodiments, the patient has a pre-vaccination lymphocyte count ≥500 cells/mm³. In other of these embodiments, the patient has a pre-vaccination lymphocyte count <500 cells/mm³.

In some embodiments, the methods comprise administering ponesimod to a patient in need thereof, administering a flu vaccine to the patient, and interrupting the administration of the ponesimod for a period of time. In certain of these embodiments, the patient has a pre-vaccination lymphocyte count ≥500 cells/mm³. In other of these embodiments, the patient has a pre-vaccination lymphocyte count <500 cells/mm³.

In some embodiments, the methods comprise interrupting ponesimod administration prior to the flu vaccine administration. In some embodiments, the ponesimod administration is interrupted for a period of time prior to the flu vaccine administration to allow for an antibody response, e.g., a positive or increased antibody concentration or an antibody response with low inhibition titers.

In some embodiments, the methods comprise interrupting the ponesimod administration for about one to four weeks prior to the flu vaccine administration. In some embodiments, the methods comprise interrupting the ponesimod administration for about four weeks starting from about one week prior to the administration of the flu vaccine. In some embodiments, the methods comprise interrupting the ponesimod administration for about three weeks starting from about one week prior to the administration of the flu vaccine.

In some embodiments, the period of time of interruption of ponesimod can be, for example, at least at least one, two, three, four, five, six, seven, eight, nine, ten, eleven, twelve, thirteen or fourteen days prior to the administration of the flu vaccine. In some embodiments, the period of time of interruption of ponesimod can be, for example, at least two, three, four, five, six, seven, eight, nine, ten, eleven or twelve weeks starting from about one or two weeks prior to the administration of the flu vaccine.

In some embodiments, the ponesimod treatment is resumed one, two, three, four, five, six, seven, eight, nine, ten, eleven, or twelve days after the flu vaccine administration. In some embodiments, the ponesimod treatment is resumed one, two, or three weeks after the flu vaccine administration.

In some embodiments, the daily dose of resumed ponesimod treatment comprises about 15 to about 25 mg of ponesimod administration. In further embodiments, the daily dose of resumed ponesimod treatment comprises about 15 mg, about 16 mg, about 17 mg, about 18 mg, about 19 mg, about 20 mg, about 21 mg, about 22 mg, about 23 mg, about 24 mg, or about 25 mg of ponesimod administration. In certain embodiments, the daily dose of resumed ponesimod treatment comprises about 20 mg of ponesimod administration.

In some embodiments, about 20 mg of ponesimod is administered orally once daily for resumed the ponesimod treatment after administration of flu vaccine. In other embodiments, the resumed ponesimod treatment after administration of flu vaccine comprises an up-titration, followed by a daily maintenance dose of ponesimod. In other embodiments, the resumed ponesimod treatment after administration of flu vaccine comprises an up-titration regimen as described herein, followed by a daily maintenance dose of ponesimod.

In certain embodiments, the flu vaccine comprises a non-live vaccine. In certain embodiments, the flu vaccine comprises an inactivated H1N1 influenza virus. In certain embodiments, the flu vaccine comprises a live vaccine.

In some embodiments, the methods comprise interrupting the ponesimod administration for about three weeks. For example, the methods comprise interrupting the ponesimod administration for about three weeks starting from about one week prior to the administration of the flu vaccine (and continuing for two weeks after the flu vaccine administration for a total of three weeks).

In some embodiments, the methods comprise interrupting the ponesimod administration for about four weeks. For example, the methods comprise interrupting the ponesimod administration for about four weeks starting from about one week prior to the administration of the flu vaccine (and continuing for three weeks after the flu vaccine administration for a total of four weeks).

In some embodiments, the methods comprise interrupting the ponesimod administration for about five weeks. For example, the methods comprise interrupting the ponesimod administration for about five weeks starting from about one week prior to the administration of the flu vaccine (and continuing for four weeks after the flu vaccine administration for a total of five weeks).

In some embodiments, the methods comprise interrupting the ponesimod administration for about six weeks. For example, the methods comprise interrupting the ponesimod administration for about six weeks starting from about one week prior to the administration of the flu vaccine (and continuing for five weeks after the flu vaccine administration for a total of six weeks).

In certain aspects, the disclosure is directed to a pharmaceutical product comprising ponesimod, wherein the pharmaceutical product is packaged, and the package includes instructions for treating MS and maintaining or maximizing flu vaccine effectiveness as described herein.

### COVID-19 Vaccine

In some embodiments, the patient to receive a COVID-19 vaccine is currently being treated with ponesimod, such that the ponesimod is administered prior to administration of the COVID-19 vaccine or concurrently with the COVID-19 vaccine.

In some embodiments, the methods comprise administering ponesimod to a patient in need thereof and administering a COVID-19 vaccine to the patient without interrupting the administration of the ponesimod for a period of time. In certain of these embodiments, the patient has a pre-vaccination lymphocyte count ≥500 cells/mm³ and/or is administered an mRNA vaccine. In other embodiments, the patient has a pre-vaccination lymphocyte count ≥500 cells/mm³ and/or is administered a viral vector vaccine. In other embodiments, the patient has a pre-vaccination lymphocyte count ≥500 cells/mm³ and/or is administered an inactivated vaccine. In other embodiments, the patient has a pre-vaccination lymphocyte count ≥500 cells/mm³ and/or is administered a protein subunit vaccine. In other embodiments, the patient has a pre-vaccination lymphocyte count >500 cells/mm³ and/or is administered vaccine different than an mRNA vaccine, for example, a viral vector vaccine, an inactivated vaccine, or protein subunit vaccine, as a first dose, and an mRNA vaccine as a second dose. In certain of these embodiments, the patient has a pre-vaccination lymphocyte count <500 cells/mm³ and/or is administered an mRNA vaccine. In other embodiments, the patient has a pre-vaccination lymphocyte count <500 cells/mm³ and/or is administered a viral vector vaccine. In other embodiments, the patient has a pre-vaccination lymphocyte count <500 cells/mm³ and/or is administered an inactivated vaccine. In other embodiments, the patient has a pre-vaccination lymphocyte count <500 cells/mm³ and/or is administered a protein subunit vaccine. In other embodiments, the patient has a pre-vaccination lymphocyte count <500 cells/mm³ and/or is administered vaccine different than an mRNA vaccine, for example, a viral vector vaccine, an inactivated vaccine, or protein subunit vaccine, as a first vaccination dose, and an mRNA vaccine as a second vaccination dose.

In some embodiments, the methods comprise administering ponesimod to a patient in need thereof, administering a COVID-19 vaccine to the patient, and interrupting the administration of the ponesimod for a period of time. In certain of these embodiments, the patient has a pre-vaccination lymphocyte count ≥500 cells/mm³ and/or is administered an mRNA vaccine. In other embodiments, the patient has a pre-vaccination lymphocyte count ≥500 cells/mm³ and/or is administered a viral vector vaccine. In other embodiments, the patient has a pre-vaccination lymphocyte count ≥500 cells/mm³ and/or is administered an inactivated vaccine. In other embodiments, the patient has a pre-vaccination lymphocyte count ≥500 cells/mm³ and/or is administered a protein subunit vaccine. In other embodiments, the patient has a pre-vaccination lymphocyte count ≥500 cells/mm³ and/or is administered vaccine different than an mRNA vaccine, for example, a viral vector vaccine, an inactivated vaccine, or protein subunit vaccine, as a first dose, and an mRNA vaccine as a second dose. In certain of these embodiments, the patient has a pre-vaccination lymphocyte count <500 cells/mm³ and/or is administered an mRNA vaccine. In other embodiments, the patient has a pre-vaccination lymphocyte count <500 cells/mm³ and/or is administered a viral vector vaccine. In other embodiments, the patient has a pre-vaccination lymphocyte count <500 cells/mm³ and/or is administered an inactivated vaccine. In other embodiments, the patient has a pre-vaccination lymphocyte count <500 cells/mm³ and/or is administered a protein subunit vaccine. In other embodiments, the patient has a pre-vaccination lymphocyte count <500 cells/mm³ and/or is administered vaccine different than an mRNA vaccine, for example, a viral vector vaccine, an inactivated vaccine, or protein subunit vaccine, as a first vaccination dose, and an mRNA vaccine as a second vaccination dose.

In some embodiments, the methods comprise interrupting ponesimod administration prior to the COVID-19 vaccine administration. In some embodiments, the ponesimod administration is interrupted for a period of time prior to the COVID-19 vaccine administration to allow for an antibody response, e.g., a positive or increased antibody concentration or an antibody response with low inhibition titers.

In some embodiments, the methods comprise interrupting the ponesimod administration for about one to four weeks prior to the COVID-19 vaccine administration. In some embodiments, the methods comprise interrupting the ponesimod administration for about four weeks starting from about one week prior to the administration of the COVID-19 vaccine. In some embodiments, the methods comprise interrupting the ponesimod administration for about three weeks starting from about one week prior to the administration of the COVID-19 vaccine.

In some embodiments, the period of time of interruption of ponesimod can be, for example, at least at least one, two, three, four, five, six, seven, eight, nine, ten, eleven, twelve, thirteen or fourteen days prior to the administration of the COVID-19 vaccine. In some embodiments, the period of time of interruption of ponesimod can be, for example, at least two, three, four, five, six, seven, eight, nine, ten, eleven or twelve weeks starting from about one or two weeks prior to the administration of the COVID-19 vaccine.

In some embodiments, the ponesimod treatment is resumed one, two, three, four, five, six, seven, eight, nine, ten, eleven, or twelve days after the COVID-19 vaccine administration. In some embodiments, the ponesimod treatment is resumed one, two, or three weeks after the COVID-19 vaccine administration.

In some embodiments, the daily dose of resumed ponesimod treatment comprises about 15 to about 25 mg of ponesimod administration. In further embodiments, the daily dose of resumed ponesimod treatment comprises about 15 mg, about 16 mg, about 17 mg, about 18 mg, about 19 mg, about 20 mg, about 21 mg, about 22 mg, about 23 mg, about 24 mg, or about 25 mg of ponesimod administration. In certain embodiments, the daily dose of resumed ponesimod treatment comprises about 20 mg of ponesimod administration.

In some embodiments, about 20 mg of ponesimod is administered orally once daily for resuming the ponesimod treatment after administration of COVID-19 vaccine. In other embodiments, the resumed ponesimod treatment after administration of COVID-19 vaccine comprises an up-titration, followed by a daily maintenance dose of ponesimod. In other embodiments, the resumed ponesimod treatment after administration of COVID-19 vaccine comprises an up-titration regimen as described herein, followed by a daily maintenance dose of ponesimod.

In certain embodiments, the COVID-19 vaccine comprises a live vaccine. In certain embodiments, the COVID-19 vaccine comprises a non-live vaccine. In certain embodiments, the non-live vaccine is an mRNA vaccine, a protein subunit vaccine, a vector vaccine or an inactivated vaccine.

In certain embodiments, the non-live COVID-19 vaccine is an mRNA vaccine.

In certain embodiments, the non-live COVID-19 vaccine is a protein subunit vaccine.

In certain embodiments, the non-live COVID-19 vaccine is a vector vaccine.

In certain embodiments, the non-live COVID-19 vaccine is an inactivated vaccine.

In certain aspects, the disclosure is directed to a pharmaceutical product comprising ponesimod, wherein the pharmaceutical product is packaged, and the package includes instructions for treating MS and maintaining or maximizing COVID-19 vaccine effectiveness as described herein.

In some embodiments, the methods include the step of determining the immune response comprising obtaining serum antibody levels in the subject elicited by the vaccine post-immunization (e.g., 4 weeks after immunization or 8 weeks post-immunization). The methods further comprise obtaining the serum antibody levels in the subject pre-immunization and comparing the serum antibody levels in the subject pre- and post-immunization.

In some embodiments, the vaccine treatment may require a booster shot. The regimens described herein, including the time periods for interruption of ponesimod before and/or after the vaccine administration would apply to such booster shots.

In some embodiments, the vaccine is administered in a single dose. In other embodiments, the vaccine is administered in multiple doses, for example, two doses or three doses. In some embodiments, the administration of the multiple doses are spaced apart by one week; or by two weeks; or by three weeks; or by four weeks; or by five weeks; or by six weeks. With respect to multiple dose vaccine administration, the period of interruption could depend on the time interval between vaccine doses. Depending on the time interval, the time periods for interruption before and/or after a vaccine dose as described herein would apply to each vaccine dose in a multiple vaccine dose administration. For example, with a vaccine requiring two doses that are six weeks apart, ponesimod administration may be interrupted about three weeks starting from about one week prior to the administration of each vaccine dose (e.g., one week before and two weeks after each vaccine dose). In another example, the multiple vaccine doses may be administered before ponesimod administration is resumed. For example, with a vaccine requiring two doses that are three weeks apart, both vaccine doses may be administered before ponesimod is resumed, such that ponesimod can be, for example, interrupted one week before the first vaccine dose and resumed two weeks after the second vaccine dose, for a total interrupted period of six weeks.

In some embodiments, the methods comprise interrupting the ponesimod administration for about three weeks. For example, the methods comprise interrupting the ponesimod administration for about three weeks starting from about one week prior to the administration of the COVID-19 vaccine (and continuing for two weeks after the COVID-19 vaccine administration for a total of three weeks).

In some embodiments, the methods comprise interrupting the ponesimod administration for about four weeks. For example, the methods comprise interrupting the ponesimod administration for about four weeks starting from about one week prior to the administration of the COVID-19 vaccine (and continuing for three weeks after the COVID-19 vaccine administration for a total of four weeks).

In some embodiments, the methods comprise interrupting the ponesimod administration for about five weeks. For example, the methods comprise interrupting the ponesimod administration for about five weeks starting from about one week prior to the administration of the COVID-19 vaccine (and continuing for four weeks after the COVID-19 vaccine administration for a total of five weeks).

In some embodiments, the methods comprise interrupting the ponesimod administration for about six weeks. For example, the methods comprise interrupting the ponesimod administration for about six weeks starting from about one week prior to the administration of the COVID-19 vaccine (and continuing for five weeks after the COVID-19 vaccine administration for a total of six weeks).

In some aspects, the methods of the disclosure are performed on a human patient having multiple sclerosis. In some embodiments, the patient's multiple sclerosis is relapsing multiple sclerosis. In other embodiments, the relapsing multiple sclerosis comprises relapsing-remitting disease, clinically isolated syndrome, or active secondary progressive disease.

As used herein, the term “ponesimod” refers to the compound (R)-5-[3-chloro-4-(2,3-dihydroxy-propoxy)-benz[Z]ylidene]-2-([Z]-propylimino)-3-o-tolyl-thiazolidin-4-one, which has the following structure:

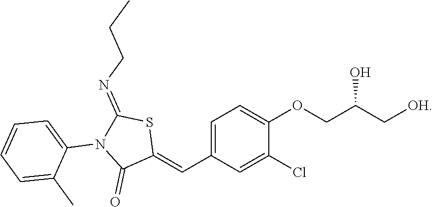 [SMILES: CCC/N=C1\S/C(=C\c2ccc(OC[C@H](O)CO)c(Cl)c2)C(=O)N1c1ccccc1C]

In some embodiments, “ponesimod” also refers to pharmaceutically acceptable salts of ponesimod. The term “pharmaceutically acceptable salt” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example Handbook of Pharmaceutical Salts. Properties, Selection and Use, P. Heinrich Stahl, Camille G. Wermuth (Eds.), Wiley-VCH, 2008 and Pharmaceutical Salts and Co-crystals, Johan Wouters and Luc Quéré (Eds.), RSC Publishing, 2012.

It is to be understood that the present disclosure encompasses ponesimod in any form including amorphous as well as crystalline forms. It is further to be understood that crystalline forms of ponesimod encompasses all types of crystalline forms including polymorphs, solvates and hydrates, salts and co-crystals (when the same molecule can be co-crystallized with different co-crystal formers) provided they are suitable for pharmaceutical administration. In some embodiments, ponesimod is in crystalline form A or crystalline form C as described in WO 2010/046835, incorporated herein by reference. In some embodiments, ponesimod is in crystalline form C.

It should be noted that the amounts of ponesimod described herein are set forth on a ponesimod free base basis. That is, the amounts indicate that amount of the ponesimod molecule administered, exclusive of, for example, solvent (such as in solvates) or counterions (such as in pharmaceutically acceptable salts).

In some embodiments, the effective regimen comprises a daily dose of ponesimod.

In some embodiments, the daily dose of ponesimod is administered orally.

In some embodiments, the daily dose of ponesimod is administered once daily.

In some embodiments, the daily dose of ponesimod is about 15 to about 25 mg. In further embodiments, the daily dose of ponesimod is about 15 mg, about 16 mg, about 17 mg, about 18 mg, about 19 mg, about 20 mg, about 21 mg, about 22 mg, about 23 mg, about 24 mg, or about 25 mg. In certain embodiments, the daily dose of ponesimod is about 20 mg.

In some embodiments, about 20 mg of ponesimod is administered orally once daily.

In other embodiments, the effective regimen comprises an up-titration, followed by a daily maintenance dose of ponesimod. An up-titration is a dosing procedure in which the daily dose of ponesimod is gradually increased over a period of days, culminating with administration of the maintenance dose.

In some embodiments, the regimen comprises an up-titration at the initiation of the method of the disclosure. In other embodiments, the regimen comprises an up-titration upon resumption of the ponesimod administration after interruption of ponesmiod administration.

In some embodiments of the methods of the disclosure, the up-titration regimen one disclosed in U.S. Pat. No. 10,220,023, incorporated herein by reference. For example, in certain aspects, the up-titration comprises administering orally once daily about 2 mg of ponesimod on days 1 and 2; about 3 mg of ponesimod on days 3 and 4; about 4 mg of ponesimod on days 5 and 6; about 5 mg of ponesimod on day 7; about 6 mg of ponesimod on day 8; about 7 mg of ponesimod on day 9; about 8 mg of ponesimod on day 10; about 9 mg of ponesimod on day 11; and about 10 mg of ponesimod on days 12, 13, 14 and/or 15.

In other embodiments of the methods of the disclosure, the up-titration comprises administering orally once daily 2 mg of ponesimod on days 1 and 2; 3 mg of ponesimod on days 3 and 4; 4 mg of ponesimod on days 5 and 6; 5 mg of ponesimod on day 7; 6 mg of ponesimod on day 8; 7 mg of ponesimod on day 9; 8 mg of ponesimod on day 10; 9 mg of ponesimod on day 11; and 10 mg of ponesimod on days 12, 13, and 14.

In some embodiments, the maintenance dose is about 20 mg of ponesimod once daily.

In some embodiments, the regimen comprises an up-titration step at initiation of the method or upon resumption of the ponesimod administration after interruption of ponesmiod administration, comprising administering orally once daily 2 mg of ponesimod on days 1 and 2; 3 mg of ponesimod on days 3 and 4; 4 mg of ponesimod on days 5 and 6; 5 mg of ponesimod on day 7; 6 mg of ponesimod on day 8; 7 mg of ponesimod on day 9; 8 mg of ponesimod on day 10; and 9 mg of ponesimod on day 11; 10 mg of ponesimod on days 12, 13, 14 and/or 15, followed by the administering of the 20 mg of ponesimod once daily thereafter. In some embodiments, 10 mg of ponesimod is administered on days 12 and 13, followed by the administering of the 20 mg of ponesimod once daily thereafter. In some embodiments, 10 mg of ponesimod is administered on days 12, 13 and 14, followed by the administering of the 20 mg of ponesimod once daily thereafter. In some embodiments, 10 mg of ponesimod is administered on days 12, 13, 14 and 15, followed by the administering of the 20 mg of ponesimod once daily thereafter.

As used herein, the term “standard of care treatment” refers to a physician-prescribed treatment of MS. In some embodiments, the standard of care comprises, consists of, or consists essentially of administering an MS treatment that has been approved by a regulatory authority. In some embodiments, the standard of care treatment is Interferon (IFN) β-1a 30 mcg i.m. once weekly (Avonex®), IFN-1a 22 or 44 mcg s.c. 3 times weekly (Rebif®), IFN β-1b 250 mcg s.c. every other day (Betaferon®, Extavia®), Pegylated IFN β-1a 125 mcg subcutaneously every 2 weeks (Plegridy®), Glatiramer acetate 20 mg s.c. once a day (o.d.) or 40 mg subcutaneously 3 times weekly (Copaxone®), Glatiramer acetate 20 mg s.c. o.d. (Glatopa®), Natalizumab 300 mg i.v. every 4 weeks (Tysabri®), Mitoxantrone i.v. every 3 months (Novantrone®), Alemtuzumab concentrate for solution for infusion, 12 mg alemtuzumab in 1.2 mL (10 mg/mL) (Lemtrada®), Fingolimod 0.5 mg orally o.d. (Gilenya®), Teriflunomide 7 mg, 14 mg o.d. (Aubagio®), Dimethyl fumarate (BG-12) gastro-resistant hard capsules 120/240 mg twice daily (Tecfidera®), or Cladribine 40 to 100 mg orally per treatment week (Mavenclad®).

In some embodiments, the standard of care treatment comprises a SIP receptor modulator that is not ponesimod.

In other embodiments, the standard of care treatment comprises fingolimod. In some embodiments, the standard of care treatment comprises administration of fingolimod orally once daily.

In some embodiments, the patient has had no prior disease modifying treatment (DMT) for multiple sclerosis. In some embodiments, the patient has had no prior disease modifying treatment (DMT) for multiple sclerosis within about two years prior to initiation of treatment with ponesimod. In some embodiments, patients that have had no prior DMT for multiple sclerosis realize improved vaccine efficacy from use of ponesimod with respect to a standard of care treatment that does not comprise ponesimod, such as fingolimod.

Accordingly, with respect to these patients and others, the disclosed methods provide health care providers with options for improved outcomes compared to standard of care.

The present disclosure also provides pharmaceutical products comprising ponesimod. Typically, the pharmaceutical product is a package or is packaged, for example, a bottle, a pouch, or a blister pack.

In some embodiments, the package includes instructions. In certain embodiments, instructions are for administering ponesimod to a human patient having multiple sclerosis in a regimen that is effective to treat multiple sclerosis and maintain or maximize vaccine effectiveness.

As used herein, unless otherwise noted, the terms “treating”, “treatment” and the like, shall include the management and care of a subject or patient (preferably mammal, more preferably human) for the purpose of combating a disease, condition, or disorder and includes the administration of a compound described herein to prevent the onset of the symptoms or complications, alleviate one or more of the symptoms or complications, or eliminate the disease, condition, or disorder.

The following Examples are provided to illustrate some of the concepts described within this disclosure. While the Examples are considered to describe certain embodiments, they should not be considered to limit the more general embodiments described herein.

### Example 1—COVID-19 Vaccine Antibody Response in RMS Patients Treated with Ponesimod

### Introduction

The effect on SARS-CoV-2 humoral response and COVID-19 infection adverse events (AEs) was characterized in RMS patients in the AC-058B202 study who have received COVID-19 vaccination while on uninterrupted ponesimod treatment.

### Methods

Participants in this study receiving 20 mg ponesimod who reported COVID-19 vaccination were analyzed. Blood samples collected before and at least 3 weeks after COVID-19 vaccination were tested for SARS-CoV-2 anti-spike-protein (spike) antibody concentrations (Human SARS-CoV-2 PreSpike IgG ELISA, Nexelis). Response to vaccination was defined as seroconversion in case of negative pre-vaccination antibody testing, or a 4-fold antibody concentration increase in case of positive pre-vaccination antibody result. COVID-19 infection adverse events (AEs) were evaluated in this study sub-population.

### Results

134 participants reported receiving COVID-19 vaccines (64.9% mRNA, 17.9% viral vector, 9.0% inactivated, 6.0% mixed, 2.2% unspecified). Median exposure to ponesimod at the time of vaccination was 10.7 (range 9.8-11.8) years. These results are summarized below in Table 1.

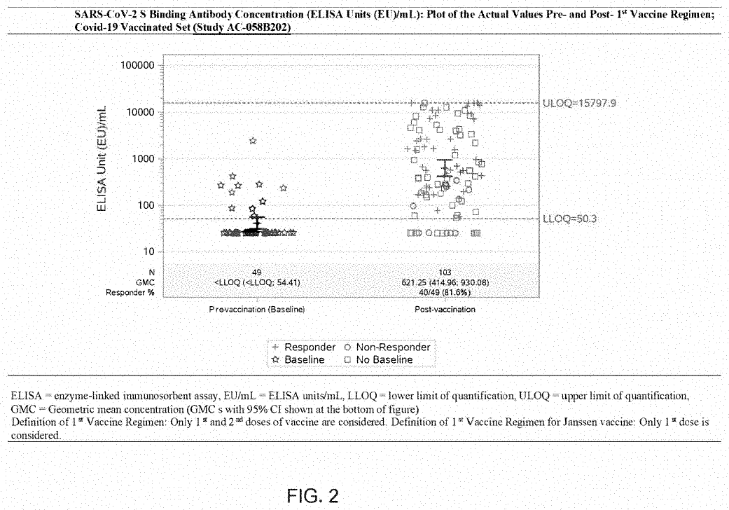

Of the 134 participants, 49 participants had both pre- and post-vaccination blood samples tested for anti-spike antibody concentrations. Out of these 49 participants, 40 (81.6%) met the pre-specified definition of response to COVID-19 vaccination. These results are summarized below in Table 2. Response was defined as pre-vaccination titer≤lower limit of quantification (LLOQ) and post-vaccination titer >LLOQ; or pre-vaccination titer >LLOQ (i.e., seroconversion in case of negative pre-vaccination antibody testing) and post-vaccination titer >4× increase (i.e., a 4-fold antibody concentration increase in case of positive pre-vaccination antibody result). These results are also shown in FIG. 2.

Of the 38 antibody-negative participants, 33 (86.8%) achieved seroconversion post-vaccination. As for clinical outcomes in the 134 vaccinated participants, 20 and 17 participants reported pre-vaccination and post-vaccination AEs of COVID-19 infection, respectively. These results are summarized below in Table 3 and Table 4.

None of these AEs were serious, severe or fatal, and none of these AEs led to permanent treatment discontinuation.

TABLE 1
TSIDEM01: Summary of Demographics and Baseline Characteristic;
Covid-19 Vaccinated Analysis Set (Study AC-0588202)

| | Ponesimod |
|---|---|
| | 20 mg |
| Analysis set: Covid-19 Vaccinated Analysis Set | 134 |
| Age at Time of Vaccination, [years] | |
| N | 134 |
| Mean (SD) | 48.5 (8.16) |
| Median | 48.5 |
| Range | (33; 65) |
| Sex | |
| N | 134 |
| Female | 89 (66.4%) |
| Male | 45 (31634) |
| Exposure (time) to ponesimod at | |
| Time of Vaccination [years] | |
| N | 134 |
| Mean (SD) | 10.73 (0.420) |
| Median | 10.71 |
| Range | (9.8; 11.8) |

TABLE 2
Summary of SARS-Cov-2 S Binding Antibody Concentration (ELISA Units (EU)/mL):
Deseriptive Statistics for 1^{st} Vaccine Regimen: Covid-19 Vaccinated Set (Study
AC-058B202)

| | Pre-vaccination (Baseline) | Post-vaccination | Fold Increase from Baseline |
|---|---|---|---|
| N | 49 | 103 | 49 |
| Geometric mean (95% CI) | <LLOQ (<LLOQ; 54.41) | 621.25 (414.96; 930.08) | 12.90 (7.64; 21.78) |
| Positive sample n (%) | 11 (22.4%) (11.77; 36.62) | 89 (86.4%)(78.25; 92.37) | |
| (95% CI) | | | |
| Responders (n/N) (%) | | | 40/49 (81.6%) (67.98; 91.24) |
| (95% CI) | | | |

Key:
CI = confidence interval; N = number of subjects with data at that time-point; For Fold Increase from Baselise: N = number of subjects with data as pre- and post-vaccination.
Exact Clopper-Pearson 95% confidence intervals are shown for Positive samples and Responders.
(LLOQ, ULOQ) (EU)/ml S-binding antibody: (50.3, 15797.9).
Data above ULOQ are imputed to ULOQ.
Positive sample refers to a quantifiable response (sample interpretation), i.e. value > LLOQ.
Responder: If the pre-vaccination value ≤ LLCO and post-vaccination value > LLOQ or if the pre-vaccination value > LLOQ and post-vaccination value > 4-fold merease.
Geometric mean concentration: values below LLOQ are imputed to LLOQ/2.
Geometric mean of fold increase-from-baseline: values below LLOQ are imputed to LLOQ.
If there are more than 1 post-vaccination samples, the last sample is used.
Percentages are calculated with the total nunber of subjects in each column as the denominator.
Definition of 1^{st} Vaccine Regimen: Only 1^{st} and 2^{nd} doses of vaccination are considered.
Definition of 1^{st} Vaccine Regimen for Janssen vaccine: Only 1^{st} dose is considered.

TABLE 3
Overall Summary of CO1D-19 Infection Adverse Events;
Covid-19 Vaccinated Analysis Set (Study AC-88202)

| | | | | >=14 D Post | >=28 D Post |
|---|---|---|---|---|---|
| | Pre-Vx | Post-Vx | Total | Vx | Vx |
| Analysis set: Covid-19 Vaccinated | | | | | |
| Analysis Set | | | 134 | | |
| Subjects with one or more COVID-19 | | | | | |
| infections, n (%) | | | | | |
| AEs | 20 (14.9%) | 17 (12.7%) | 33 (24.6%) | 17 (12.7%) | 17 (12.7%) |
| AEs leading to death^{a} | 0 | 0 | 0 | 0 | 0 |
| Serious AEs | 0 | 0 | 0 | 0 | 0 |
| AE: leading to treatssent isteraption | 5 (3.7%) | 4 (3.0%) | 7 (5.2%) | 4 (3.0%) | 4 (3.0%) |
| AEs leading to treatseat discontinuation | 0 | 0 | 0 | 0 | 0 |
| Severity | | | | | |
| Mild | 18 (13.4%) | 13 (9.7%) | 28 (20.9%) | 13 (9.7%) | 13 (9.7%) |
| Moderate | 2 (1.5%) | 4 (3.0%) | 6 (4.5%) | 4 (3.0%) | 4 (3.0%) |
| Severe | 0 | 0 | 0 | 0 | 0 |

Key:
AS = adverse event.
>=14 D Post Vx: At least 14 days post-vaccination (after 1^{st} dose of vaccination).
>=28 D Post Vx: At least 28 days post-vaccination (after 1^{st} dose of vaccination).
^{a}AEs leading to death are based on AE outcome of Fatal.
Percentages are calculated based on the total number of subjects in the Covid-19 vaccinated analysis set as the denominator

TABLE 4
Summary of SARS-CoV-2 S Binding Antibody Concentration
(ELISA Units (EU)/mL); Descriptive Statistics for 1^{st} Vaccine
Regimen: Pre- and Post -Vaccination;
Covid-19 Vaccinated Set (Study AC-058B202)

| | Pre-vaccination | Pre-vaccination |
|---|---|---|
| | Negative | Positive |
| Number of subjects wish both | 38 | 11 |
| pre- and post-vaccination samples | | |
| Post-vaccination N (%) | | |
| Negative | 5 (13.2%) | 0 |
| Positive | 33 (86.8%) | 11 (100.0%) |
| Responder | 33 (86.8%) | 7 (63.6%) |

Key:
Positive sample refers to a quantifiable response (sample interpretation); i.e. value > LLOQ
Responder: If the pre-vaccination value ≤ LLOQ and post-vaccination value > LLOQ or if the pre-vaccination value > LLOQ and post-vaccination value > 4-fold increase.
If there are more than 1 post-vaceination samples, the last sample is used.
Percentages are calculated with the total number of subjects in each column as the denominator.
Definition of 1^{st} Vaccine Regimen: Only 1^{st} and 2^{nd} doses of vaccination are considered.
Defntion of 1^{st} Vaccine Regimen for Janssen vaccine: Only 1^{st} dose is considered.

### Conclusion

In patients with RMS on ponesimod treatment, the majority appears to develop a measurable SARS-CoV-2 humoral response after COVID-19 vaccination.

### Example 2—COVID-19 Antibody Response by Vaccine Type and Lymphocyte Count in RMS Patients Treated with Ponesimod

### Introduction

The SARS-CoV-2 humoral response by vaccine type and pre-vaccination lymphocyte count in RMS patients who have received COVID-19 vaccination while on ponesimod treatment was investigated.

### Methods

Patients in the Phase 2 extension study AC-058B202 receiving ponesimod 20 mg who reported COVID-19 vaccination were included in this analysis. Blood samples collected before and at least 3 weeks after vaccination were evaluated with Human SARS-CoV-2 PreSpike IgG (spike antibody) enzyme-linked immunoassay (Nexelis). A responder to vaccination was defined as seroconversion in case of negative pre-vaccination antibody testing, or a 4-fold antibody concentration increase in case of positive pre-vaccination antibody result. Responder analysis was performed by vaccine type, and by pre-vaccination lymphocyte count.

### Results

49 COVID-19-vaccinated patients had both pre- and post-vaccination blood samples available for analysis. These patients were on uninterrupted ponesimod treatment during the course of vaccination. Response to COVID-19 vaccination varied by vaccination type: of 32 participants who received mRNA vaccines, 29 (90.6%) met responder criteria. Of 7 participants who received viral vector vaccines, 5 (71.4%) were responders. Of 6 participants who received inactivated vaccines, 2 (33.3%) were responders. Of 4 participants who received mixed or unspecified vaccine types, all 4 (100%) were responders. Response to COVID-19 vaccination also varied by lymphocyte count: of 16 participants with pre-vaccination lymphocyte count <500 cells/mm³, 9 (56.3%) were responders. Of 32 participants with lymphocyte count ≥500 cells/mm³, 30 (93.8%) were responders.

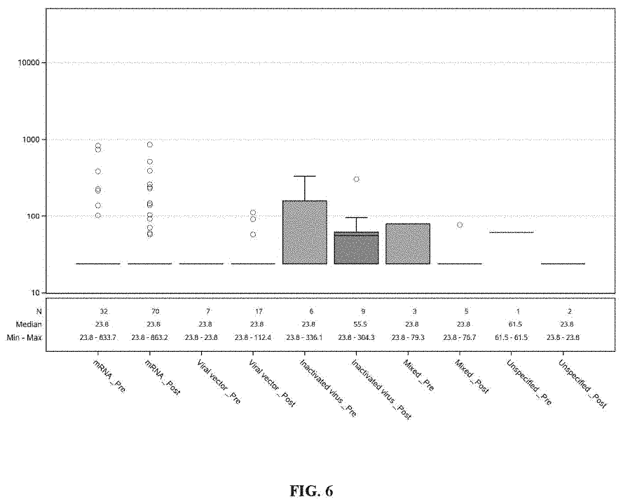

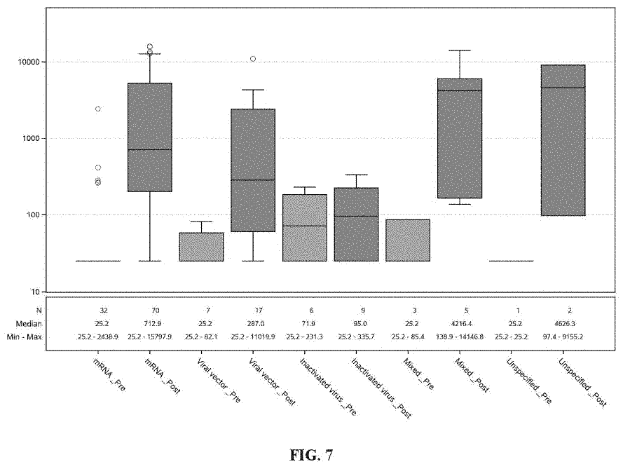

Results are further supported by Table 5 below and FIGS. 6 and 7.

In FIG. 6, ELISA=enzyme-linked immunosorbent assay, EU/mL=ELISA units/mL, LLOQ=lower limit of quantification, ULOQ=upper limit of quantification;

N=number of participants with available values in the specified group; and

Y-axis=Actual values are displayed on a log 10 scale;

The boxes represent the 25th and 75th percentile, the whiskers represent the minimum and maximum of the non-outliers. Outliers are defined as values beyond 1.5 times the interquartile range. The lines represent the median.
Definition of 1^{st} Vaccine Regimen: Only 1^{st} and 2^{nd} doses of vaccine are considered.
Definition of 1^{st} Vaccine Regimen for Janssen vaccine: Only 1^{st} dose is considered.

In FIG. 7, ELISA=enzyme-linked immunosorbent assay, EU/mL=ELISA units/mL, LLOQ=lower limit of quantification, ULOQ=upper limit of quantification;

N=number of participants with available values in the specified group; and

Y-axis=Actual values are displayed on a log 10 scale.

The boxes represent the 25th and 75th percentile, the whiskers represent the minimum and maximum of the non-outliers. Outliers are defined as values beyond 1.5 times the interquartile range. The lines represent the median.
Definition of 1^{st} Vaccine Regimen: Only 1^{st} and 2^{nd} doses of vaccine are considered.
Definition of 1^{st} Vaccine Regimen for Janssen vaccine: Only 1^{st} dose is considered.

TABLE 5
Summary of SARS-CoV-2 S Binding Antibody Concentration (ELISA Units (EU)/mL):
Descriptive Statistics by Vaccine Type for 1^{st} Vaccine Regimen; Covid-19 Vaccinated Set
(Study AC-058B202)

| | Pre-vaccination (Baseline) | Post-vaccination | Fold Increase from Baseline |
|---|---|---|---|
| mRNA vaccines | | | |
| N | 32 | 70 | 32 |
| Geometric mean | <LLOQ (<LLOQ; 59.37) | 833.22 (513.88; 1351.00) | 20.68 (11.35; 37.68) |
| (95% CI) | | | |
| Positive sample n (%) | 5 (15.6%) (5.28; 32.79) | 62 (88.6%) (78.72; 94.93) | |
| (95% CI) | | | |
| Responders (n/N) (%) | | | 29/32 (90.6%) (74.98; 98.02) |
| (95% CI) | | | |
| Viral vector vaccines | | | |
| N | 7 | 17 | 7 |
| Geometric mean | <LLOQ (<LLOQ; 53.36) | 390.49 (140.01; 1089.07) | 7.76 (2.22; 27.11) |
| (95% CI) | | | |
| Positive sample n (%) | 2 (28.6%) (3.67; 70.96) | 14 (82.4%) (56.57; 96.2) | |
| (95% CI) | | | |
| Responders (n/N) (%) | | | 5/7 (71.4%) (29.04; 96.33) |
| (95% CI) | | | |
| Inactivated virus vaccines | | | |
| N | 6 | 9 | 6 |
| Geometric mean | 65.88 (<LLOQ; 203.92) | 84.22 (<LLOQ; 189.22) | 1.29 (0.50; 3.32) |
| (95% CI) | | | |
| Positive sample n (%) | 3 (50.0%) (11.81; 88.19) | 6 (66.7%) (29.93; 92.51) | |
| (95% CI) | | | |
| Responders (n/N) (%) | | | 2/6 (33.3%) (4.33; 77.72) |
| (95% CI) | | | |
| Mixed vaccines | | | |
| N | 3 | 5 | 3 |
| Geometric mean | <LLOQ | 1525.40 | 11.49 |
| (95% CI) | | | |
| Positive sample n (%) | 1 (33.3%) | 5 (100.0%) | |
| (95% CI) | | | |
| Responders (n/N) (%) | | | 3/3 (100.0%) |
| (95% CI) | | | |
| Unspecified vaccines | | | |
| N | 1 | 2 | 1 |
| Geometric mean | <LLOQ | 944.31 | 182.01 |
| (95% CI) | | | |
| Positive sample n (%) | 0 | 2 (100.0%) | |
| (95% CI) | | | |
| Responders (n/N) (%) | | | 1/1 (100.0%) |
| (95% CI) | | | |

Key:
CI = confidence interval; N = number of subjects with data at that time-point; For Fold Increase from Baseline: N = number of subjects with data at pre- and post-vaccination.
Exact Clopper-Pearson 95% confidence intervals are shown for Positive samples and Responders.
Confidence Intervals are provided only when n >= 6 or if not all values are equal.
(LLOQ, ULOQ) (EU)/mL: S-binding antibody: (50.3, 15797.9).
Data above ULOQ are imputed to ULOQ.
Positive sample refers to a quantifiable response (sample interpretation); i.e. value > LLOQ.
Responder: If the pre-vaccination value ≤ LLOQ and post-vaccination value > LLOQ or if the pre-vaccination value > LLOQ and post-vaccination value > 4-fold increase.
Geometric mean concentration: values below LLOQ are imputed to LLOQ/2.
Geometric mean of fold increase-from-baseline: values below LLOQ are imputed to LLOQ.
If there are more than 1 post-vaccination samples, the last sample is used.
Percentages are calculated with the total number of subjects in each column and category as the denominator.
Definition of 1^{st} Vaccine Regimen: Only 1^{st} and 2^{nd} doses of vaccine are considered.
Definition of 1^{st} Vaccine Regimen for Janssen vaccine: Only 1^{st} dose is considered.

### Conclusion

Humoral response to COVID-19 vaccines varied by vaccine type and pre-vaccination lymphocyte count. In ponesimod-treated patients who received mRNA COVID-19 vaccines and/or who had pre-vaccination lymphocyte counts ≥500 cells/mm³, most (>90%) met antibody concentration responder criteria.

### Example 3—Ponesimod and the Restoration of the Ability to Mount Post-Vaccination Antibodies

### Introduction

The effect of ponesimod treatment on the response to vaccination in mice without anti-vaccine immunity was investigated, and whether there is a benefit from a drug holiday around vaccination.

### Methods

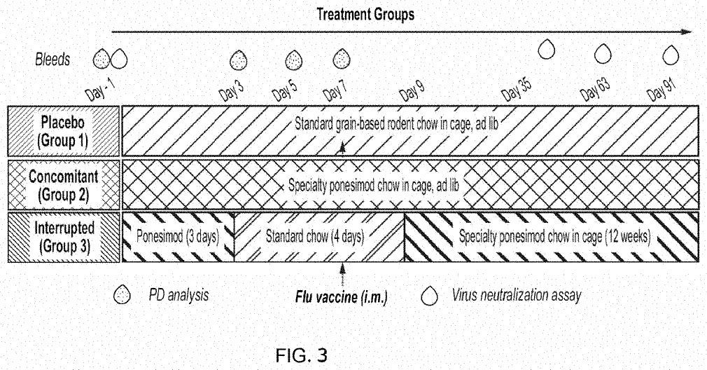

The neutralizing antibody response to a single intramuscular injection of purified and inactivated influenza A/PR/8/34 was compared in female BALB/c mice that were maintained on different oral ponesimod regimens (Group 1: standard chow; Group 2: continuous specialty chow with ponesimod at 1000 ppm; Group 3: specialty chow with a 4-day discontinuation around immunization). Treatment groups are summarized in FIG. 3.

### Results

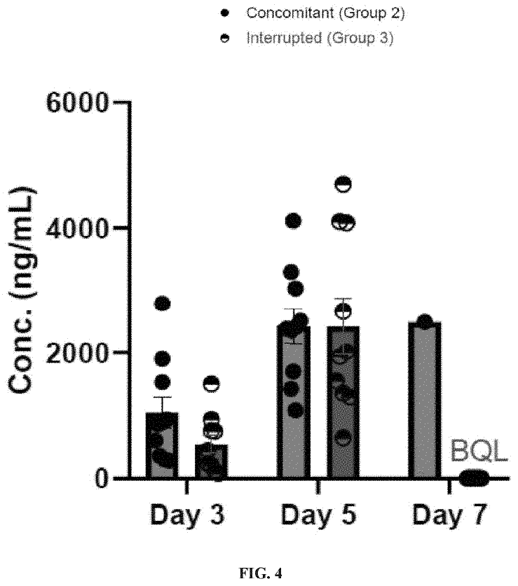

Plasma drug levels were sufficiently high to reduce lymphocyte counts by Day 5 in Groups 2 and 3, but after a 2-day wash-out period, ponesimod was cleared from plasma in Group 3, as shown in FIG. 4. Drug levels were variable between animals, likely because of different chow consumption prior to sample collection. Data points are values from individual mice; Bars show mean±SEM and BQL is below quantitation limit.

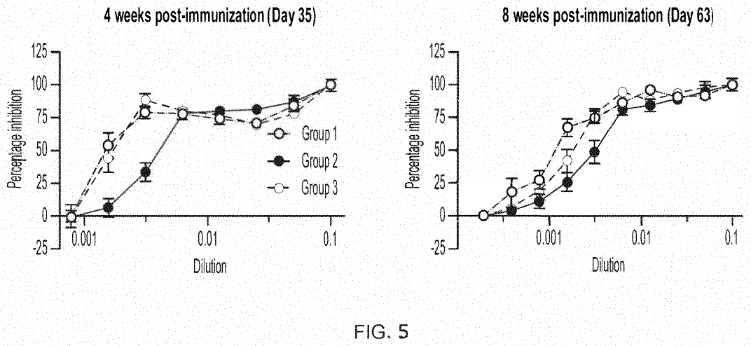

Animals in Group 2 that were continuously maintained on ponesimod chow, including at the time of flu vaccine administration, had an attenuated vaccine response compared to control mice (Group 1). A brief drug discontinuation (Group 3) allowed for the formation of an antibody response with low inhibition titers (i.e., very effective virus neutralization), as shown in FIG. 5. Mean percentage inhibition of viral infection by A/PR/8/34 with serum samples, ±SEM.

Table 5 presents data regarding dilution from neat serum that results in 50% inhibition of viral infection (ID50). Lower numbers represent more effective viral inhibition.

TABLE 5
Data Summary—Inhibition Titer

| Time Point | Placebo | Concomitant | Discontinued |
|---|---|---|---|
| (Days) | (Group 1) | (Group 2) | (Group 3) |
| −1 | — | 0.050 | 0.001 |
| 35 | 0.001 | 0.004 | 0.001 |
| 63 | 0.001 | 0.003 | 0.002 |
| 91 | 0.002 | 0.004 | 0.003 |

### Conclusions

Mice treated with ponesimod had a blunted, but not absent, antibody response to the inactivated H1N1 influenza virus. Briefly halting ponesimod treatment helped in achieving maximal vaccination effectiveness.

What is claimed:

1. A method of treating multiple sclerosis (MS) and maximizing vaccine effectiveness, comprising:
administrating ponesimod to a patient in need thereof,
administering a vaccine to the patient, and
interrupting the administration of the ponesimod for a period of time.

2. The method of claim 1, wherein the ponesimod is administered prior to or concurrently with the administration of the vaccine.

3. The method of claim 1, wherein the ponesimod administration is interrupted prior to the administration of the vaccine and resumed after a period of time following the vaccine administration.

4. A method of treating multiple sclerosis (MS) and maintaining vaccine effectiveness, comprising:
administrating ponesimod to a patient in need thereof; and
administering a vaccine to the patient.

5. The method of claim 4, wherein the ponesimod is administered prior to or concurrently with the administration of the vaccine.

6. The method of claim 4, wherein the ponesimod is administered prior to the administration of the vaccine.

7. The method of claim 4, wherein the method comprises no interruption of ponesimod administration prior to or after the vaccine administration.

8. The method of claim 1, wherein the ponesimod administration is interrupted for a period of time to allow for an antibody response.

9. The method of claim 8, wherein the antibody response is a positive or increased antibody concentration or an antibody response with low inhibition titers.

10. The method of claim 1, wherein the ponesimod administration is interrupted for about one to seven days.

11. The method of claim 10, wherein the ponesimod administration is interrupted for about 4 days.

12. The method of claim 1, wherein the ponesimod administration is interrupted for about six weeks.

13. The method of claim 12, wherein the ponesimod administration is interrupted for about six weeks starting from about one week prior to the administration of the vaccine.

14. The method of claim 1, wherein the ponesimod administration is interrupted for about five weeks.

15. The method of claim 14, wherein the ponesimod administration is interrupted for about five weeks starting from about one week prior to the administration of the vaccine.

16. The method of claim 1, wherein the ponesimod administration is interrupted for about four weeks.

17. The method of claim 16, wherein the ponesimod administration is interrupted for about four weeks starting from about one week prior to the administration of the vaccine.

18. The method of claim 1, wherein the ponesimod administration is interrupted for about three weeks.

19. The method of claim 18, wherein the ponesimod administration is interrupted for about three weeks starting from about one week prior to the administration of the vaccine.

20. The method of claim 8, wherein the administration of ponesimod is resumed after the interruption.

21. The method of claim 1, wherein about 20 mg of ponesimod is administered orally once daily.

22. The method of claim 20, wherein the resumption of the ponesimod administration comprises an up-titration, comprising administering orally once daily 2 mg of ponesimod on days 1 and 2; 3 mg of ponesimod on days 3 and 4; 4 mg of ponesimod on days 5 and 6; 5 mg of ponesimod on day 7; 6 mg of ponesimod on day 8; 7 mg of ponesimod on day 9; 8 mg of ponesimod on day 10; and 9 mg of ponesimod on day 11; 10 mg of ponesimod on days 12, 13, and 14, followed by administering 20 mg of ponesimod once daily thereafter.

23. The method of claim 1, wherein the MS is relapsing multiple sclerosis.

24. The method of claim 23, wherein the relapsing multiple sclerosis comprises relapsing-remitting disease, clinically isolated syndrome, or active secondary progressive disease.

25. The method of claim 1, wherein the vaccine is a flu vaccine.

26. The method of claim 25, wherein the flu vaccine comprises an inactivated H1N1 influenza virus.

27. The method of claim 1, wherein the vaccine is a COVID-19 vaccine.

28. The method of claim 27, wherein the COVID-19 vaccine comprises a non-live vaccine.

29. The method of claim 28, wherein the non-live vaccine is an mRNA vaccine, a protein subunit vaccine, a vector vaccine, or an inactivated vaccine.

30. The method of claim 28, wherein the non-live vaccine is an mRNA vaccine.

31. A pharmaceutical product comprising ponesimod, wherein the pharmaceutical product is packaged, and the package includes instructions for treating MS and maintaining or maximizing vaccine effectiveness.

32. The pharmaceutical product of claim 31, wherein the vaccine is a COVID-19 vaccine or a flu vaccine.

33. The pharmaceutical product of claim 31, wherein the vaccine is a COVID-19 vaccine.

34. The pharmaceutical product of claim 31, wherein the vaccine is a flu vaccine.

35. The method of claim 1, wherein the patient has a pre-vaccination lymphocyte count <500 cells/mm³.

36. The method of claim 1, wherein the patient has a pre-vaccination lymphocyte count ≥500 cells/mm³.
